(19) United States
(12) Patent Application Publication
(10) Pub. No.: US 2016/0063183 A1
(43) Pub. Date: Mar. 3, 2016

(54) MEDICAL INFORMATION DISTRIBUTION SYSTEM

(71) Applicants: Kabushiki Kaisha Toshiba, Minato-ku (JP); Toshiba Medical Systems Corporation, Otawara-shi (JP)

(72) Inventors: Yoshinori Shimizu, Nasushiobara (JP); Masahiro Ozawa, Sakura (JP)

(73) Assignees: Kabushiki Kaisha Toshiba, Minato-ku (JP); Toshiba Medical Systems Corporation, Otawara-shi (JP)

(21) Appl. No.: 14/938,510

(22) Filed: Nov. 11, 2015

Related U.S. Application Data
(63) Continuation of application No. PCT/JP2014/063435, filed on May 21, 2014.

(30) Foreign Application Priority Data
May 21, 2013 (JP) 2013-107293

(51) Int. Cl. G06F 19/00; A61B 6/00

(52) U.S. Cl. CPC G06F 19/32; A61B 6/463; A61B 6/485

(57) ABSTRACT

According to one embodiment, a medical information distribution system includes a display controller, a display content setting unit, and an information transmitter. The display controller displays a plurality of pieces of medical information from medical equipment or peripheral equipment on a display of the medical equipment or a display of each of a plurality of devices. The display content setting unit sets display content selected from the pieces of medical information based on the identification information of each of the medical equipment and the devices. The information transmitter sends the display content of the pieces of medical information to the medical equipment and the devices.

### CROSS-REFERENCE TO RELATED APPLICATION

This application is based upon and claims the benefit of priority from Japanese Patent Application No. 2013-107293, filed May 21, 2013; the entire contents of which are incorporated herein by reference.

### FIELD

Embodiments described herein relate generally to a medical information distribution system.

### BACKGROUND

So-called hybrid approach has recently been introduced, which combines traditional surgical and interventional catheterization procedures to perform them in parallel. In the control room and the examination room where the hybrid approach is carried out, for example, a large display having a screen divided into a plurality of areas or a plurality of small displays are installed. A variety of medical information is displayed on these displays. Examples of the medical information include X-ray fluoroscopic images generated by an X-ray diagnosis apparatus, X-ray images (road map images) in which the blood vessel is emphasized by a contrast agent, a camera image of the incision captured by an optical camera, biological wave information such as electrocardiographic (ECG) waveform, respiratory waveform, and brain waves, X-ray irradiation information that indicates whether X-rays are generated in the X-ray diagnosis apparatus, and peripheral equipment information that indicates ultrasound images generated by an ultrasound diagnosis apparatus located near the X-ray diagnosis apparatus, the operation of an anesthesia machine, and the like.

On the other hand, the hybrid approach involves a variety of medical staff such as, for example, catheterization specialist, surgeon, anesthesiologist, biological wave information observer, and nursing staff. They work together in the examination room and the control room to perform their individual roles.

In such a case, for example, desired medical information to be displayed on the display may vary depending on individual medical staff. Besides, for example, the medical staff who works away from the display may have a difficulty in checking medical information displayed on the display as being interrupted by other medical staff.

### BRIEF DESCRIPTION OF THE DRAWINGS

FIG. 1 is a block diagram of an X-ray diagnosis apparatus and peripheral equipment according to an embodiment;

FIG. 2 is a schematic view of the X-ray diagnosis apparatus of the embodiment;

FIG. 3 is a flowchart of the operation of the embodiment;

FIG. 4 is a schematic view of an image displayed on a display of the embodiment;

FIG. 5 is a conceptual diagram of distribution content information of the embodiment;

FIG. 6A is a schematic view of an image displayed on the display of the embodiment;

FIG. 6B is a schematic view of an image displayed on the display of the embodiment; and

FIG. 7 is a schematic view of an examination room and a control room of the embodiment.

### DETAILED DESCRIPTION

In general, according to one embodiment, a medical information distribution system includes a display controller, a display content setting unit, and an information transmitter. The display controller displays a plurality of pieces of medical information from medical equipment or peripheral equipment on a display of the medical equipment or a display of each of a plurality of devices. The display content setting unit sets display content selected from the pieces of medical information based on the identification information of each of the medical equipment and the devices. The information transmitter sends the display content of the pieces of medical information to the medical equipment and the devices.

Referring now to the drawings, illustrative embodiments are described below.

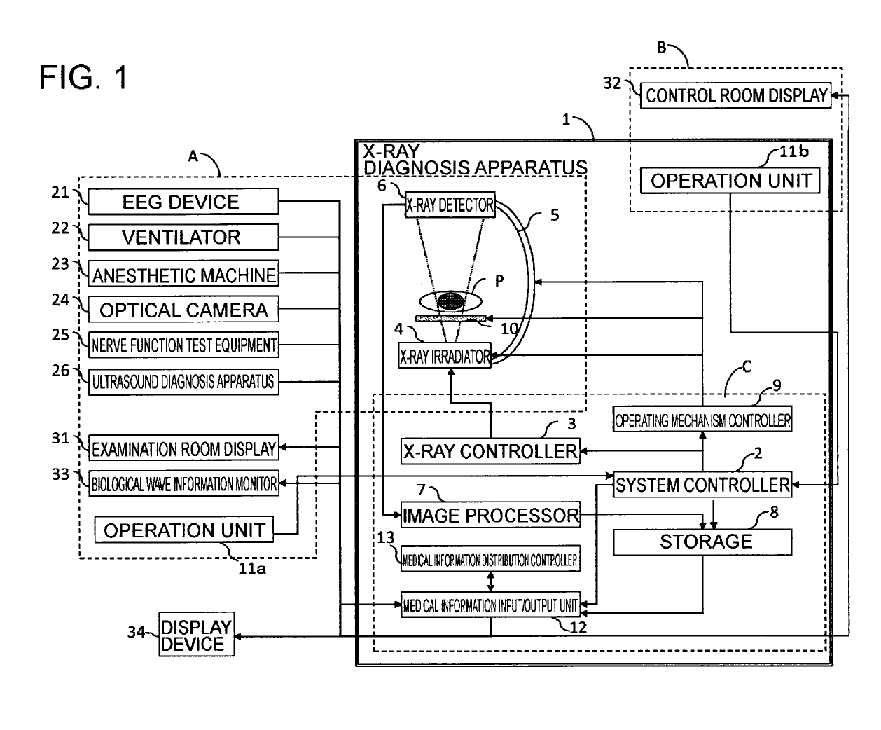

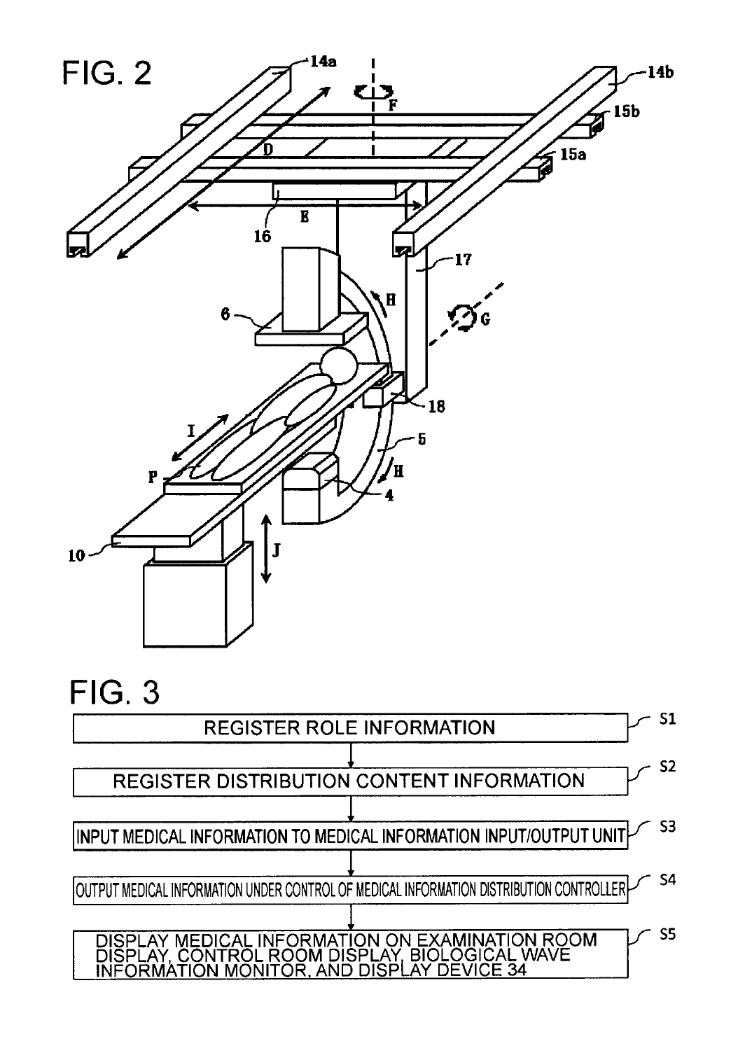

In the following, an X-ray diagnosis apparatus is described as a medical information distribution system of the embodiment. First, with reference to FIGS. 1 and 2, a description is given of the configuration of an X-ray diagnosis apparatus 1 and peripheral equipment of the embodiment, and also a relationship therebetween.

FIG. 1 is a block diagram of the X-ray diagnosis apparatus 1 and the peripheral equipment of the embodiment. FIG. 2 is a schematic view of the X-ray diagnosis apparatus of the embodiment. In FIG. 1, portions enclosed by dotted lines A, B, and C are provided to an examination room, a control room, and a machine room, respectively.

As illustrated in FIG. 1 or 2, the X-ray diagnosis apparatus 1 of this embodiment includes a system controller 2, an X-ray controller 3, an X-ray irradiator 4, a C-arm 5, an X-ray detector 6, an image processor 7, a storage 8, an operating mechanism controller 9, a bed 10, an operation unit 11a, an operation unit 11b, a medical information input/output unit 12, a medical information distribution controller 13, a rail 14a, a rail 14b, a rail 15a, a rail 15b, a base 16, a support post 17, and a C-arm support 18. The X-ray irradiator 4, the C-arm 5, the X-ray detector 6, the bed 10, the operation unit 11a, the rail 14a, the rail 14b, the rail 15a, the rail 15b, the base 16, the support post 17, and the C-arm support 18 are provided to the examination room. The system controller 2, the X-ray controller 3, the image processor 7, the storage 8, the operating mechanism controller 9, the medical information input/output unit 12, and the medical information distribution controller 13 are provided to the machine room. Besides, the operation unit 11b is provided to the control room.

The system controller 2 instructs the X-ray controller 3 according to an instruction provided by the operator through the operation unit 11a or 11b. The system controller 2 feeds the medical information input/output unit 12 with a medical image stored in the storage 8. The system controller 2 generates a predetermined operation screen at a predetermined timing, and feeds it to the medical information input/output unit 12. Having instructed the X-ray controller 3 to irradiate X-rays according to an instruction provided by the operator through the operation unit 11a or 11b, the system controller 2 feeds X-ray irradiation information to the medical information input/output unit 12. Having instructed the X-ray controller 3 to stop the X-ray irradiation according to an instruction provided by the operator through the operation unit 11a or 11b, the system controller 2 instructs the medical information input/output unit 12 to stop distributing the X-ray irradiation information. In this embodiment, the system controller 2 instructs the operating mechanism controller 9 according to an instruction provided by the operator through the operation unit 11a or 11b.

The X-ray controller 3 includes a high-voltage generator, and applies a voltage to an X-ray tube of the X-ray irradiator 4 according to an instruction from the system controller 2. The X-ray controller 3 stops the application of the voltage to the X-ray irradiator 4 according to an instruction from the system controller 2.

The X-ray irradiator 4 includes the X-ray tube and an X-ray diaphragm. The X-ray tube irradiates X-rays when a voltage is applied thereto by the high-voltage generator of the X-ray controller 3. The X-ray diaphragm adjusts the irradiation range of X-rays irradiated from the X-ray tube according to an instruction from the operating mechanism controller 9.

The X-ray detector 6 detects X-rays irradiated from the X-ray irradiator 4 and having transmitted through a patient P placed on the bed 10. The X-ray detector 6 generates a detection signal based on the X-rays detected, and sends the detection signal to the image processor 7.

The image processor 7 generates medical image data based on the detection signal received from the X-ray detector 6. Examples of the medical image data include X-ray fluoroscopic image data and road map image data. For example, the image processor 7 sends X-ray fluoroscopic image data and road map image data thus generated to the storage 8.

The storage 8 stores the medical image data received from the image processor 7. The storage 8 feeds the medical image data to the medical information input/output unit 12 according to an instruction from the system controller 2.

The rails 14a and 14b are supported by, for example, the ceiling of the examination room, and are configured to move the rails 15a and 15b in directions indicated by arrow D in FIG. 2 according to an instruction from the operating mechanism controller 9. The rails 15a and 15b are configured to move the base 16 in directions indicated by arrow E in FIG. 2 according to an instruction from the operating mechanism controller 9. The base 16 is configured to rotate the support post 17 in directions indicated by arrow F in FIG. 2 according to an instruction from the operating mechanism controller 9. The support post 17 is configured to rotate the C-arm support 18 in directions indicated by arrow G in FIG. 2 according to an instruction from the operating mechanism controller 9. The C-arm support 18 causes the C-arm 5 to slide in a direction indicated by arrow H in FIG. 2 according to an instruction from the operating mechanism controller 9. The C-arm 5 holds the X-ray irradiator 4 and the X-ray detector 6 at its ends such that they face each other.

The operating mechanism controller 9 controls the operation of the X-ray diaphragm according to an instruction from the system controller 2, and controls the irradiation range of X-rays. The operating mechanism controller 9 instructs the rails 14a, 14b, 15a and 15b, the base 16, the support post 17, and the C-arm support 18 on their operation according to an instruction from the system controller 2 to change the position and angle of the X-ray irradiator 4 and the X-ray detector 6. The operating mechanism controller 9 instructs the bed 10 on its operation according to an instruction from the system controller 2 to move the position of the patient P placed on the bed 10.

The bed 10 is used to place the patient P, and moves the position of the patient P in, for example, directions indicated by arrows I and J in FIG. 2 according to an instruction from the operating mechanism controller 9.

The operation unit 11a includes, for example, a lever or a switch. The operator provides instructions to the system controller 2 through the operation unit 11a. Examples of the instructions include instructions to irradiate X-rays and to stop the X-ray irradiation, and an instruction to the operating mechanism controller 9. The operation unit 11b includes, for example, a lever, a switch, a mouse and a keyboard. The operator provides instructions to the system controller 2 through the operation unit 11b. Examples of the instructions include instructions to irradiate X-rays and to stop the X-ray irradiation, an instruction to the operating mechanism controller 9, and an instruction related to a predetermined operation screen displayed on the display used in the control room.

The electroencephalograph (EEG) device 21 is peripheral equipment to measure the brain waves of the patient P. The EEG device 21 feeds the medical information input/output unit with the brain waves measured as biological wave information.

A ventilator 22 is peripheral equipment to measure the respiratory waves of the patient P. The ventilator 22 feeds the medical information input/output unit 12 with the breathing waves measured as biological wave information.

An anesthesia machine 23 is peripheral equipment, and feeds the input to the medical information input/output unit 12 with, for example, the amount of anesthetic gas used for the patient P as peripheral equipment information.

An optical camera 24 is peripheral equipment photograph, for example, the incision of the patient P in a surgery and generates camera image data. The optical camera 24 feeds the camera image data to the medical information input/output unit 12.

Nerve function test equipment 25 is peripheral equipment to measure the ECG waveform of the patient P. The nerve function test equipment 25 feeds the medical information input/output unit 12 with the ECG waveform measured as biological wave information.

An ultrasound diagnosis apparatus 26 is peripheral equipment to generate ultrasound diagnostic image data of the patient P. The ultrasound diagnosis apparatus 26 feeds the medical information input/output unit 12 with the ultrasound diagnostic image data as peripheral equipment information.

An examination room display 31 is installed in the examination room as an output unit. The examination room display 31 includes a display unit for displaying medical information output from the medical information input/output unit 12 for distribution.

A control room display 32 is installed in the control room as an output unit. The control room display 32 includes a display unit for displaying medical information output from the medical information input/output unit 12 for distribution.

A biological wave information monitor 33 is installed in the examination room as an output unit. The biological wave information monitor 33 includes a display unit for displaying medical information output from the medical information input/output unit 12 for distribution.

A display device 34 is, for example, a tablet that is communicable with the X-ray diagnosis apparatus via radio or wire, and is a user interface having an input unit and a display unit. The display device 34 is portable, and is carried as an output unit by medical staff in the examination room or the control room. It is assumed herein that, for example, the medical staff who uses the display device 34 is a nurse. The display device 34 registers role information according to input provided by the medical staff through the input unit. The display device 34 notifies the medical information distribution controller 13 of the role information thus registered. The display device 34 displays medical information output from the medical information input/output unit 12 for distribution. The role information is described in detail later.

The medical information input/output unit 12 is fed with medical information from the inside of the X-ray diagnosis apparatus 1 such as, for example, the system controller 2 and the storage 8. In addition, the medical information input/output unit 12 is fed with medical information from the outside of the X-ray diagnosis apparatus 1 such as, for example, the EEG device 21, the ventilator 22, the anesthesia machine 23, the optical camera 24, the nerve function test equipment 25, and the ultrasound diagnosis apparatus 26. The medical information input/output unit 12 outputs the medical information to the outside such as, for example, the examination room display 31, the control room display 32, the biological wave information monitor 33, and the display device 34, according to an instruction from the medical information distribution controller 13.

The medical information distribution controller 13 instructs the medical information input/output unit 12 on medical information to be output to the examination room display 31, the control room display 32, and the biological wave information monitor 33 based on preset information registered in advance. The preset information is used to determine the medical information to be output by the medical information input/output unit 12 to the examination room display 31, the control room display 32, and the biological wave information monitor 33. The medical information distribution controller 13 instructs the medical information input/output unit 12 to output information to the examination room display 31 based on the preset information. Examples of the information include the X-ray irradiation information received from the system controller 2, the X-ray fluoroscopic image data and the road map image data received from the storage 8, the biological wave information received from the EEG device 21, the ventilator 22 and the nerve function test equipment 25, the peripheral equipment information received from the anesthesia machine 23 and the ultrasound diagnosis apparatus 26, and the camera image data received from the optical camera 24. The medical information distribution controller 13 instructs the medical information input/output unit 12 to output information to the control room display 32 based on the preset information. Examples of the information include the X-ray irradiation information received from the system controller 2, the road map image data received from the storage 8, and the camera image data received from the optical camera 24. The medical information distribution controller 13 instructs the medical information input/output unit 12 to output information to the biological wave information monitor 33. Examples of the information include the biological wave information received from the EEG device 21, the ventilator 22 and the nerve function test equipment 25.

Besides, the medical information distribution controller 13 registers distribution content information based on the role information notified by the display device 34. The medical information distribution controller 13 instructs the medical information input/output unit 12 on the medical information to be output to the display device 34 based on the distribution content information thus registered. The distribution content information is described in detail later.

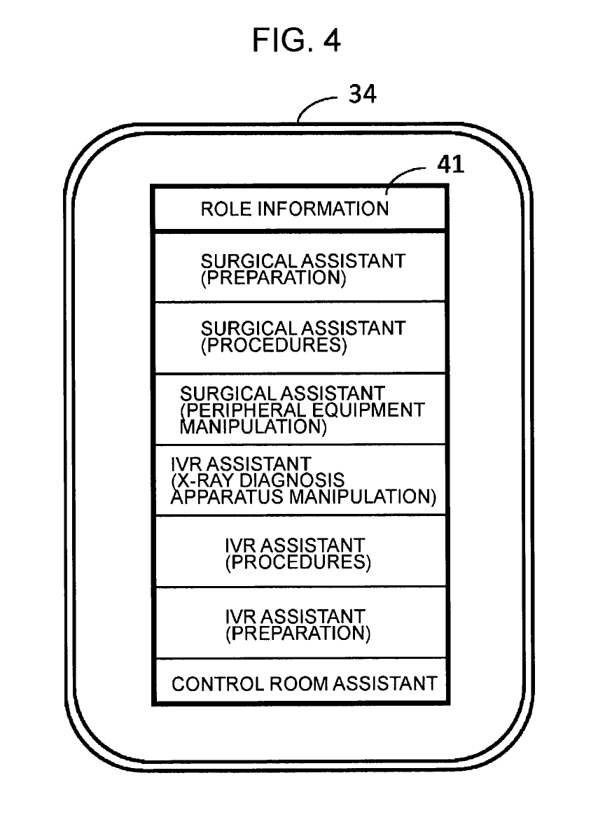

Next, with reference to FIGS. 3 to 6, a description is given of a flow of the medical information fed to and output from the medical information input/output unit 12 of this embodiment.

FIG. 3 is a flowchart illustrating a flow of the medical information fed to and output from the medical information input/output unit 12 of this embodiment. Incidentally, in FIG. 3, steps S1 and S2 represent a process before examination starts, and step S3 and subsequent steps represent a process after the examination starts.

In step S1, the nurse who uses the display device 34 activates the display device 34, and registers role information.

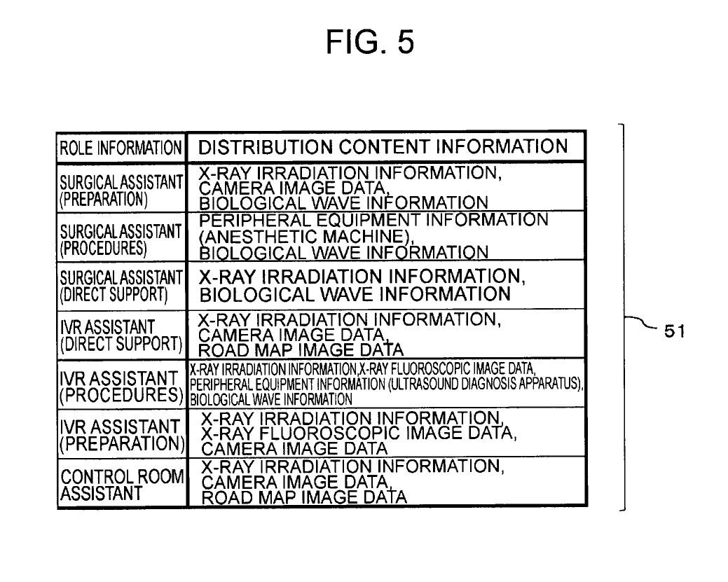

FIG. 4 is a schematic diagram illustrating an example of a screen displayed on the display unit of the display device 34 at the start-up of the display device 34. As illustrated in FIG. 4, at this time, role information columns 41 are displayed on the screen of the display unit of the display device 34. The role information columns 41 indicate a plurality of pieces of role information such as, for example, surgical assistant for preparation, surgical assistant for procedures, surgical assistant for direct support, interventional radiology (IVR) assistant for direct support, IVR assistant for procedures, IVR assistant for preparation, control room assistant, and the like. Incidentally, a person in charge of surgical assistant for preparation is in a role of, for example, preparing medical equipment such as a scalpel and medicament to be used. A person in charge of surgical assistant for procedures is in a role of, for example, supporting a surgery with an anesthesia machine or the like. A person in charge of surgical assistant for direct support is in a role of, for example, passing medical equipment such as a scalpel to a surgeon. A person in charge of IVR assistant for direct support is in a role of, for example, passing medical equipment such as a catheter to an IVR specialist. A person in charge of IVR assistant for procedures is in a role of, for example, supporting IVR procedures using an ultrasound diagnosis apparatus or the like. A person in charge of IVR assistant for preparation is in a role of, for example, preparing medical equipment such as a catheter and medicament to be used. A person in charge of control room assistant is in a role of, for example, supporting overall surgery and IVR procedures in the control room.

When a screen as illustrated in FIG. 4 is displayed on the display unit, the nurse who uses the display device 34 selects role information corresponding to his/her role using the input unit of the display device 34. When role information is selected by the nurse who uses the display device 34, the display device 34 registers the role information selected as role information that corresponds to the role of the nurse who uses the display device 34. The display device 34 notifies the medical information distribution controller 13 of the role information thus registered.

In step S2, having been notified of the role information, the medical information distribution controller 13 registers the distribution content information.

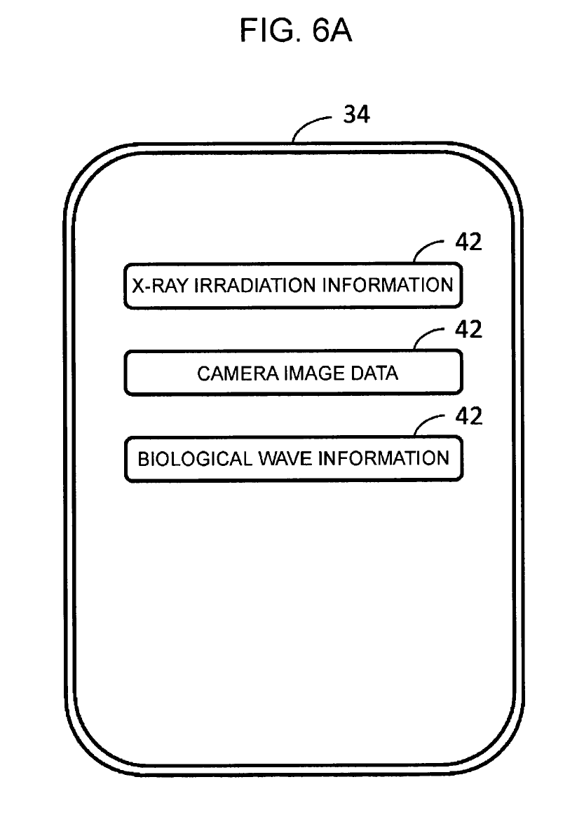

FIG. 5 is a schematic diagram of a distribution content setting table 51 used for convenience to describe the registration of the distribution content information. The medical information distribution controller 13 registers the distribution content information corresponding to the display device 34 based on, for example, the distribution content setting table 51 and the role information notified by the display device 34.

For example, if the role information notified by the display device 34 indicates surgical assistant for preparation, the medical information distribution controller 13 registers X-ray irradiation information, camera image data information, and biological wave information as the distribution content information corresponding to the display device 34. This is because the nurse in charge of surgical assistant for preparation needs a camera image to check the progress of a surgery in preparation for surgical instruments such as a scalpel, the biological wave information to prepare medicament conforming to the condition of the patient, and the X-ray irradiation information to avoid exposure to X-rays as much as possible.

It is assumed herein that the distribution content includes, in addition to medical information such as biological information, camera image data information, and X-ray irradiation information (described later), information on the type, display size, and display position of the medical information. Incidentally, the information on the type, display size, and display position of the medical information may be set in advance according to the role information (including operator information and operator location information).

For example, if the role information notified by the display device 34 indicates surgical assistant for procedures, the medical information distribution controller 13 registers peripheral equipment information and biological wave information as the distribution content information corresponding to the display device 34. This is because the nurse in charge of surgical assistant for procedures needs, for example, the peripheral equipment information to know the state of peripheral equipment to be used and the biological wave information to know the condition of the patient during a surgery.

For example, if the role information notified by the display device 34 indicates surgical assistant for direct support, the medical information distribution controller 13 registers X-ray irradiation information and biological wave information as the distribution content information corresponding to the display device 34. This is because the nurse in charge of surgical assistant for direct support needs the X-ray irradiation information to avoid exposure to X-rays as much as possible and the biological wave information to know the condition of the patient during a surgery.

For example, if the role information notified by the display device 34 indicates IVR assistant for direct support, the medical information distribution controller 13 registers X-ray irradiation information, camera image data, and road map image data as the distribution content information corresponding to the display device 34. This is because the nurse in charge of IVR assistant for direct support needs the X-ray irradiation information to avoid exposure to X-rays as much as possible, and the camera image data as well as the road map image data to pass a catheter or the like used in IVR to a doctor.

For example, if the role information notified by the display device 34 indicates IVR assistant for procedures, the medical information distribution controller 13 registers X-ray irradiation information, X-ray fluoroscopic image data, peripheral equipment information, and biological wave information as the distribution content information corresponding to the display device 34. This is because the nurse in charge of IVR assistant for procedures needs the X-ray irradiation information to avoid exposure to X-rays as much as possible, the X-ray fluoroscopic image data to observe the state of the procedures, the peripheral equipment information to know the state of peripheral equipment to be used, and the biological wave information to know the condition of the patient during a surgery.

For example, if the role information notified by the display device 34 indicates IVR assistant for preparation, the medical information distribution controller 13 registers X-ray irradiation information, X-ray fluoroscopic image data, and camera image data information as the distribution content information corresponding to the display device 34. This is because the nurse in charge of IVR assistant for preparation needs the X-ray irradiation information to avoid exposure to X-rays as much as possible, the X-ray fluoroscopic image data to observe the state of the procedures to prepare equipment and chemical solution to be used along with the progress of the procedures, and the camera image data to check the progress of a surgery as being at a distance from the patient.

For example, if the role information notified by the display device 34 indicates control room assistant, the medical information distribution controller 13 registers X-ray irradiation information, camera image data, and road map image data as the distribution content information corresponding to the display device 34. This is because the nurse in charge of control room assistant needs, for example, the X-ray irradiation information, the camera image data, and the road map image data to roughly know the situation taking place in the examination room.

In step S3, after an examination is initiated, the medical information input/output unit 12 is fed with a variety of medical information.

In step S4, the medical information distribution controller 13 instructs the medical information input/output unit 12 on the medical information to be output to the display device 34 based on preset information registered in advance and the distribution content information registered in step S2.

For example, if the nurse who uses the display device 34 is in charge of surgical assistant for preparation, according to an instruction from the medical information distribution controller 13, the medical information input/output unit 12 outputs, to the display device 34, the X-ray irradiation information received from the system controller 2, the camera image data received from the optical camera 24, and the biological wave information received from the EEG device 21, the ventilator 22, and the nerve function test equipment 25.

For example, if the nurse who uses the display device 34 is in charge of surgical assistant for procedures, according to an instruction from the medical information distribution controller 13, the medical information input/output unit 12 outputs, to the display device 34, the peripheral equipment information received from the anesthesia machine 23 and the ultrasound diagnosis apparatus 26, and the biological wave information received from the EEG device 21, the ventilator 22 and the nerve function test equipment 25.

For example, if the nurse who uses the display device 34 is in charge of surgical assistant for direct support, according to an instruction from the medical information distribution controller 13, the medical information input/output unit 12 outputs, to the display device 34, the X-ray irradiation information received from the system controller 2, and the biological wave information received from the EEG device 21, the ventilator 22 and the nerve function test equipment 25.

For example, if the nurse who uses the display device 34 is in charge of IVR assistant for direct support, according to an instruction from the medical information distribution controller 13, the medical information input/output unit 12 outputs, to the display device 34, the X-ray irradiation information received from the system controller 2, the road map image data received from the storage 8, and the camera image data received from the optical camera 24.

For example, if the nurse who uses the display device 34 is in charge of IVR assistant for procedures, according to an instruction from the medical information distribution controller 13, the medical information input/output unit 12 outputs, to the display device 34, the X-ray irradiation information received from the system controller 2, the X-ray fluoroscopic image data received from the storage 8, the peripheral equipment information received from the ultrasound diagnosis apparatus 26, and the biological wave information received from the EEG device 21, the ventilator 22 and the nerve function test equipment 25.

For example, if the nurse who uses the display device 34 is in charge of IVR assistant for preparation, according to an instruction from the medical information distribution controller 13, the medical information input/output unit 12 outputs, to the display device 34, the X-ray irradiation information received from the system controller 2, the X-ray fluoroscopic image data received from the storage 8, and the camera image data received from the optical camera 24.

For example, if the nurse who uses the display device 34 is in charge of control room assistant, according to an instruction from the medical information distribution controller 13, the medical information input/output unit 12 outputs, to the display device 34, the X-ray irradiation information received from the system controller 2, the road map image data received from the storage 8, and the camera image data received from the optical camera 24.

Meanwhile, according to an instruction from the medical information distribution controller 13, the medical information input/output unit 12 outputs, to the examination room display 31, the X-ray irradiation information received from the system controller 2, the X-ray fluoroscopic image data and the road map image data received from the storage 8, the biological wave information received from the EEG device 21, the ventilator 22 and the nerve function test equipment 25, the peripheral equipment information received from the anesthesia machine 23 and the ultrasound diagnosis apparatus 26, and the camera image data received from the optical camera 24. Besides, according to an instruction from the medical information distribution controller 13, the medical information input/output unit 12 outputs, to the control room display 32, the X-ray irradiation information received from the system controller 2, the road map image data received from the storage 8, and the camera image data received from the optical camera 24. Further, according to an instruction from the medical information distribution controller 13, the medical information input/output unit 12 outputs the biological wave information received from the EEG device 21, the ventilator 22 and the nerve function test equipment 25 to the biological wave information monitor 33.

In step S5, the examination room display 31, the control room display 32, the biological wave information monitor 33, and the display device 34 each display the medical information output thereto.

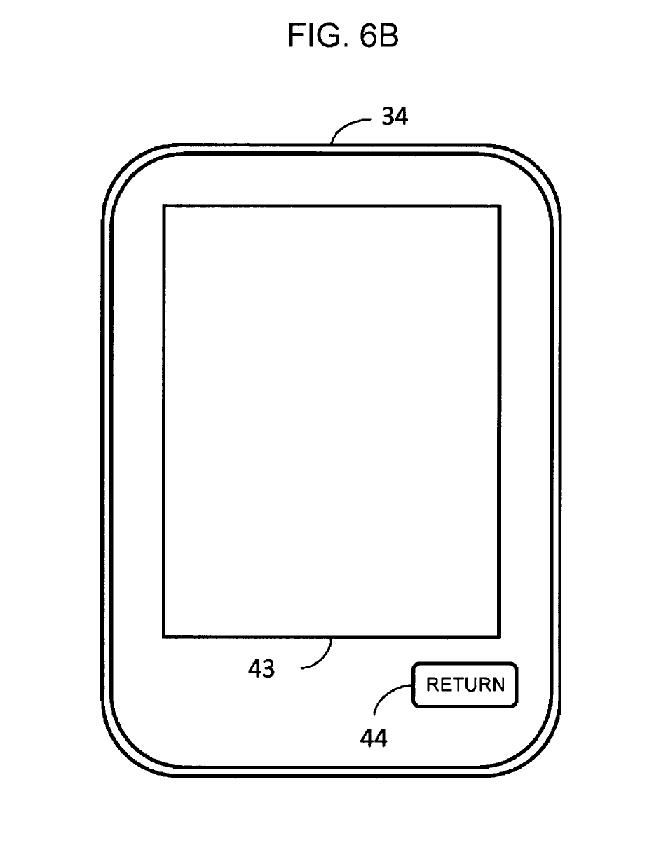

FIGS. 6A and 6B are schematic diagrams each illustrating a screen displayed on the display unit of the display device 34 when the medical information is output from the medical information input/output unit 12. For example, it is assumed herein that the nurse who uses the display device 34 is in charge of surgical assistant for preparation.

When the medical information is output from the medical information input/output unit 12, first, the display device 34 displays a screen as illustrated in FIG. 6A. As can be seen in FIG. 6A, the screen displays medical information selection buttons 42 corresponding to the types of the medical information output from the medical information input/output unit 12 to the display device 34. The display unit of the display device 34 displays, as illustrated in FIG. 6A, the medical information selection buttons 42 corresponding to the X-ray irradiation information, the camera image data, and the biological wave information. The nurse who uses the display device 34 selects one of the medical information selection buttons 42 displayed on the display unit of the display device 34 through the input unit of the display device 34. When one of the medical information selection buttons 42 is selected by the nurse who uses the display device 34, the screen of the display device 34 is switched to a screen as illustrated in FIG. 6B, which displays a medical information display field 43. The medical information display field 43 displays the medical information output from the medical information input/output unit 12 to the display device 34, which corresponds to the selected one of the medical information selection buttons 42. Incidentally, if the nurse who uses the display device 34 selects a return button 44 displayed besides the medical information display field 43, the screen is switched to the one as illustrated in FIG. 6A to allow the user to refer to other medical information.

As described above, in the X-ray diagnosis apparatus 1 of the embodiment, the medical information input/output unit 12 collectively manages medical information generated by the X-ray diagnosis apparatus 1 and the peripheral equipment, and, according to an instruction from the medical information distribution controller 13, outputs it to a predetermined output unit. With this, for example, even if the examination room display 31 and the control room display 32 display medical information that is not desired by the medical staff who uses the display device 34, the display device 34 displays desired medical information. Thereby, the medical staff can refer to the desired information. Besides, for example, when the medical staff who uses the display device 34 is working away from the examination room display 31, the control room display 32 and the biological wave information monitor 33, and is having a difficulty in checking desired medical information displayed thereon as being interrupted by other medical staff and medical equipment, he/she can refer to the desired medical information displayed on the display device 34. That is, even if a plurality of medical staff are working together, each of them can check appropriate medical information.

In this embodiment, while a description is given by taking the role information and the distribution content information illustrated in FIG. 5 as an example, the operator may arbitrarily change these pieces of information or make an addition thereto. For example, role information corresponding to the role of other medical staff, such as surgeon, surgical physician assistant, IVR specialist and IVR physician assistant, may be added to the role information. Besides, for example, medical image data, such as X-ray CT images and MRI images, may be acquired from a picture archiving and communication system (PACS), and added to the distribution content information. Alternatively, image supplementary information, such as patient ID, name, and the date of birth, may be acquired from a hospital information system (HIS) or a radiology information system (RIS), and added to the distribution content information.

In this embodiment, an example is described in which a single medical staff uses one display device (34); however, there may be a plurality of display devices (34), and a plurality of medical staff use them. For example, if all medical staff in the examination room and the control room use the display device 34, the same medical information as displayed on the display device 34 need not necessarily be displayed on the examination room display 31, the control room display 32, and the biological wave information monitor 33. Accordingly, the examination room display 31, the control room display 32, and the biological wave information monitor 33 can be downsized. In addition, the space created by the downsizing of the examination room display 31, the control room display 32, and the biological wave information monitor 33 is available for other purposes.

In this embodiment, an example is described in which the display device 34 registers role information in advance, and the medical information input/output unit 12 distributes medical information corresponding to the role information registered; however, for example, the X-ray diagnosis apparatus 1 may obtain the position of the display device 34 to automatically set the role according to the position of the display device 34 in the examination room and the control room. This allows the medical staff who uses the display device 34 to refer to medical information conforming to the location.

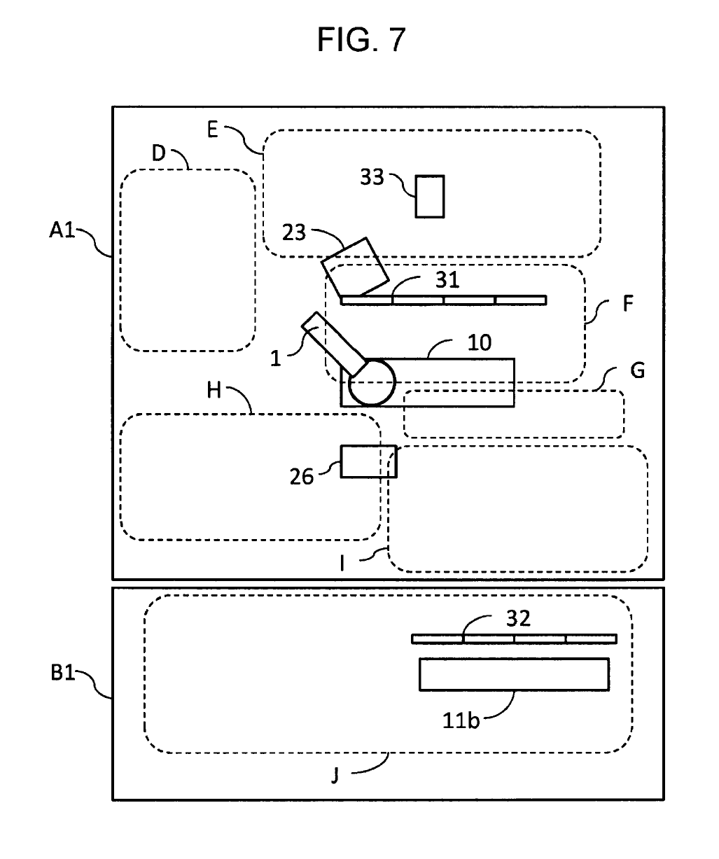

FIG. 7 is a schematic diagram of an examination room A and a control room B. As illustrated in FIG. 7, an examination room A1 (an area surrounded by dotted line A in FIG. 1) includes areas D, E, F, G, H, and I each surrounded by a dotted line. Besides, the control room B1 (an area surrounded by dotted line B in FIG. 1) includes an area J. For example, if the display device 34 is located in the area D, the role of the medical staff who uses the display device 34 is set to surgical assistant for preparation. For example, if the display device 34 is located in the area E, the role of the medical staff who uses the display device 34 is set to surgical assistant for procedures. For example, if the display device 34 is located in the area F, the role of the medical staff who uses the display device 34 is set to surgical assistant for direct support. For example, if the display device 34 is located in the area G, the role of the medical staff who uses the display device 34 is set to IVR assistant for direct support. For example, if the display device 34 is located in the area H, the role of the medical staff who uses the display device 34 is set to IVR assistant for procedures. For example, if the display device 34 is located in the area I, the role of the medical staff who uses the display device 34 is set to IVR assistant for preparation. For example, if the display device 34 is located in the area J, the role of the medical staff who uses the display device 34 is set to control room assistant.

For example, in the areas D, E, F, G, H, and I, there may be arranged a stand where the display device 34 can be placed. In this case, when the display device 34 is placed on the stand in each area, role information corresponding to the area may be registered in the medical information distribution controller 13 as the role information of the display device 34.

In this embodiment, an example is described in which the distribution content information corresponding to the role information is fixed regardless of the progress of surgery or IVR procedures; however, it may be automatically changed (updated) according to the progress of surgery or IVR procedures. This makes it possible to distribute medical information more suitable to the medical staff who uses the display device 34.

Besides, the distribution content information corresponding to the role information may be automatically changed (updated) according to the condition of the subject, and the display content on the examination room display 31, the control room display 32, the biological wave information monitor 33, the display device 34, or the like may also be changed (updated). The condition of the subject includes the biological condition of the subject such as, for example, heart rate, oxygen concentration, blood pressure of the subject along with the start of a transfusion, and blood pressure of the subject along with the start of cardiopulmonary bypass.

In this embodiment, an example is described in which the medical staff who uses the display device 34 registers the role information; however, for example, the operator of the X-ray diagnosis apparatus 1 may register the role information through the operation unit 11b. At this time, the operator may also register the correspondence relationship between the role information and the distribution content information. In this case, for example, the distribution content setting table 51 is displayed on the control room display. The operator enters information to the distribution content setting table 51 through the operation section 11b.

In this embodiment, an example is described in which medical information to be output to the examination room display 31, the control room display 32, and the biological wave information monitor 33 is determined based on the preset information registered in advance; however, it may be determined based on the role information and the distribution content information like medical information to be output to the display device 34. In this case, the role information and the distribution content information are determined based on, for example, the installation location of the examination room display 31, the control room display 32 and the biological wave information monitor 33, or the medical staff who refers to information displayed on the examination room display 31, the control room display 32 and the biological wave information monitor 33. In this case, for example, the role information and the distribution content information are registered through the operation portion 11b of the X-ray diagnosis apparatus 1 as described above.

In this embodiment, an example is described in which, as illustrated in FIG. 6B, the display device 34 displays medical information one by one on the screen; however, it may display a plurality of pieces of medical information at the same time by dividing the screen. In this case, the medical staff who uses the display device 34 can refer to the pieces of medical information without operating the display device 34, thereby dispensing with the trouble of operation.

In this embodiment, an example is described in which the medical information from the peripheral equipment is directly input to the medical information input/output unit 12; however, it may be once stored in the storage 8, and then output therefrom to the medical information input/output unit 12.

In this embodiment, an example is described in which the X-ray diagnosis apparatus 1 includes the medical information input/output unit 12 and the medical information distribution controller 13; however, the medical information input/output unit 12 and the medical information distribution controller 13 may be separately provided as a medical information distribution device. In this case, by connecting the medical information distribution device to the X-ray diagnosis apparatus not having the medical information input/output unit 12 and the medical information distribution controller 13, the same functions as described in the embodiment can be achieved.

Further, while this embodiment is described by taking the X-ray diagnosis apparatus 1 as an example including the medical information input/output unit 12 and the medical information distribution controller 13 which constitute the medical information distribution system, it is not a limitation. The embodiment may also be applicable to, for example, MRI apparatus and ultrasound diagnosis apparatus.

While certain embodiments have been described, these embodiments have been presented by way of example only, and are not intended to limit the scope of the inventions. Indeed, the novel embodiments described herein may be embodied in a variety of other forms; Further, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information distribution system comprising processing circuitry configured to:
display a plurality of pieces of medical information from medical equipment or peripheral equipment on a display of the medical equipment or a display of each of a plurality of devices;
set display content selected from the pieces of medical information based on identification information of each of the medical equipment and the devices; and
send the display content of the pieces of medical information to the medical equipment and the devices.

2. The medical information distribution system of claim 1, wherein the identification information is based on a role of medical staff who refers to information displayed on the medical equipment and the devices.

3. The medical information distribution system of claim 1, wherein the identification information is based on a location of each of the medical equipment and the devices.

4. The medical information distribution system of claim 1, wherein the processing circuitry is further configured to update the display content according to progress of an examination determined based on information from the medical equipment or the peripheral equipment.

5. The medical information distribution system of claim 1, wherein the processing circuitry is further configured to update the display content based on conditions of a subject.

6. The medical information distribution system of claim 1, wherein, the display content includes information on setting of type, display size, and display position of the medical information to be displayed.

7. The medical information distribution system of claim 1, wherein the medical information includes at least one of X-ray irradiation information, camera image data, biological wave information, peripheral equipment information, road map image data, and X-ray fluoroscopic image data.
